(19) United States
(12) Patent Application Publication
(10) Pub. No.: US 2013/0074844 A1
(43) Pub. Date: Mar. 28, 2013

(54) USE OF MULTIPLE BREATH TYPES

(71) Applicants: Gardner Kimm, Carlsbad, CA (US); Peter Doyle, Vista, CA (US)

(72) Inventors: Gardner Kimm, Carlsbad, CA (US); Peter Doyle, Vista, CA (US)

(73) Assignee: Nellcor Puritan Bennett LLC, Boulder, CO (US)

(21) Appl. No.: 13/241,799

(22) Filed: Sep. 23, 2011

(51) Int. Cl. A61M 16/00

(52) U.S. Cl. 128/204.23; 128/204.21

(57) ABSTRACT

The present disclosure describes a mode of ventilation that makes an automatic determination of an appropriate mandatory breath type in response to one or more patient based criteria. Specifically, the ventilator during the delivery a mandatory breath type determines whether predetermined ventilatory criteria have been met. Based on the determination, the ventilator may deliver one of any number of mandatory breath types. Further, the present disclosure also combines the advantages of a hybrid mode of ventilation with this automatic determination of an appropriate mandatory breath type in response to one or more patient based criteria.

### INTRODUCTION

Medical ventilator systems have long been used to provide ventilatory and supplemental oxygen support to patients. These ventilators typically comprise a source of pressurized oxygen which is fluidly connected to the patient through a conduit or tubing. As each patient may require a different ventilation strategy, modern ventilators can be customized for the particular needs of an individual patient. For example, several different ventilator modes and breath types have been created to provide better ventilation for patients in various different scenarios.

### USE OF MULTIPLE BREATH TYPES

This disclosure describes systems and methods for ventilating a patient with new modes of ventilation. The disclosure describes a novel mode for selecting a breath type from existing breath types for the patient based on monitored patient parameters.

One aspect of the disclosure relates to a method for operating a ventilator, The method includes:
a) receiving a user selection of two or more mandatory breath types from a plurality of mandatory breath types;
b) monitoring one or more patient respiratory parameters during ventilation of a patient;
c) comparing at least one monitored patient respiratory parameter to at least one predetermined mandatory threshold; and
d) delivering a mandatory breath of a selected one of the two or more selected mandatory breath types to the patient based on results of the mandatory comparing operation.

Yet another aspect of the disclosure relates to a ventilator system that includes at least one processor and at least one memory. The memory is communicatively coupled to the at least one processor and contains instructions for a plurality of breath types and instructions for operating a ventilator in at least an adjusting mandatory mode that, when executed by the at least one processor, performs a method. The method includes:
a) monitoring one or more patient respiratory parameters during ventilation of a patient;
b) comparing at least one monitored patient respiratory parameter to at least one predetermined mandatory threshold;
c) selecting one of a preselected set of mandatory breath types based on results of the mandatory comparing operation; and
d) delivering a mandatory breath of the selected one of the preselected set of mandatory breath types to the patient.

An additional aspect of the disclosure relates to a graphical user interface for a ventilator. The ventilator is configured with a computer having a user interface including the graphical user interface for accepting commands. The graphical user interface includes at least one window associated with the graphical user interface and one or more elements within the at least one window, The one or more elements include at least one of a mode button allowing selection of one of a plurality of modes and a mandatory breath type selection element through which a plurality of mandatory breath types is selected to be delivered to a patient.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings,

It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of described technology and are not meant to limit the scope of the invention as claimed in any manner, which scope shall be based on the claims appended hereto.

FIG. 1 is a diagram illustrating an embodiment of an exemplary ventilator connected to a human patient.

FIG. 2 is a block-diagram illustrating an embodiment of a ventilatory system having a user interface for operating a ventilator.

FIG. 3 illustrates an embodiment of method for ventilating a patient with a Mandatory Adjusting Mode.

FIG. 4 illustrates an embodiment of method for ventilating a patient with an Adjusting Hybrid Mode in a mandatory adjust setting.

FIG. 5 illustrates an embodiment of method for ventilating a patient with an Adjusting Hybrid Mode in a dual adjust setting.

FIG. 6 is an illustration of a user interface for setting up a new patient attached for ventilation.

### DETAILED DESCRIPTION

For the purposes of this disclosure, a “breath” refers to single cycle of inspiration and exhalation delivered with the assistance of a ventilator. The term “breath type” refers to some specific definition or set of rules dictating how the pressure and flow of respiratory gas is controlled by the ventilator during a breath. Breath types may be mandatory breath types (that is, the delivery of the breath is initiated by the ventilator and/or patient) or spontaneous (which refers to breath types in which the breath is initiated only by the patient).

A ventilation “mode”, on the other hand, is a set of rules controlling how multiple subsequent breaths should be delivered. Modes may be mandatory, that is controlled by the ventilator, or spontaneous, that is that allow a breath to be delivered or controlled upon detection of a patient's effort to inhale, exhale or both. For example, a simple mandatory mode of ventilation is to deliver one breath of a specified mandatory breath type at a clinician-selected respiratory rate (e.g., one breath every 6 seconds). Until the mode is changed, ventilators will continue to provide breaths of the specified breath type as dictated by the rules defining the mode. This specification describes novel modes of ventilation, such as an Adjusting Mandatory Mode, an Adjusting Spontaneous Mode, and an Adjusting Hybrid Mode. Further, the Adjusting Hybrid Mode may contain three different settings, such as mandatory adjust, spontaneous adjust, and dual adjust.

Different mandatory breath types suit different patient scenarios. Oftentimes, just using one mandatory breath type may cause the patient to receive an inappropriate breath (such as an insufficient tidal volume). When only one mandatory breath type is delivered to the patient, the clinician must become aware that the patient is being delivered an inappropriate breath and then change the ventilator settings to an appropriate mandatory breath type. The present disclosure introduces a method for automatically determining which mandatory breath type, of a plurality of mandatory breath types, should be delivered to a patient during an Adjusting Mandatory Mode.

Specifically, the ventilator detects that patient measurements have exceeded or fallen below a predetermined threshold associated with patient based criteria. When the ventilator determines that the threshold has been crossed, the ventilator delivers the appropriate mandatory breath type, avoiding many of the pitfalls experienced by previous ventilators that were limited to a Mandatory Mode that delivers only a single mandatory breath type until clinician action is taken.

Further, different spontaneous breath types suit different patient scenarios. Oftentimes, just using one spontaneous breath type may cause the patient to receive an insufficient size of breath (tidal volume). The clinician must become aware that the patient is being delivered an insufficient amount of breath and then change the ventilator settings to an appropriate spontaneous breath type. The present disclosure introduces a method for automatically determining which spontaneous breath type, of a plurality of spontaneous breath types, should be delivered to a patient during an Adjusting Spontaneous Mode. Specifically, the ventilator detects that patient measurements have exceeded or fallen below the predetermined threshold associated with patient based criteria. When the ventilator determines that the threshold has been crossed, the ventilator delivers the appropriate spontaneous breath type, avoiding many of the pitfalls experienced by previous ventilator Spontaneous Modes that deliver only a single spontaneous breath type until clinician action is taken,

Additionally, Hybrid Modes have been previously utilized to determine if the ventilator should deliver a selected spontaneous breath type in a spontaneous mode or if the ventilator should deliver a selected mandatory breath type in a mandatory mode. The ventilator delivers the spontaneous breath type if a patient inspiratory effort is detected within a predetermined time period or backup rate and delivers a mandatory breath type if a patient inspiratory effort is not detected within the predetermined time period or backup rate during the Hybrid Mode. However, oftentimes, just using one spontaneous breath type and/or one mandatory breath type causes the patient to receive an insufficient and/or inappropriate breath in a Hybrid Mode. The clinician must become aware that the patient is being delivered an insufficient and/or inappropriate breath and then change the ventilator settings to an appropriate spontaneous and/or mandatory breath type. The present disclosure introduces a method for automatically determining when a spontaneous breath type should be delivered, of a plurality of spontaneous breath types when a patient effort is detected and/or when a mandatory breath type should be delivered, of a plurality of mandatory breath types when a patient trigger is not detected during an Adjusting Hybrid Mode. Specifically, the ventilator detects that patient measurements have exceeded or fallen below predetermined thresholds associated with patient based criteria. When the ventilator determines that a threshold has been crossed, the ventilator delivers the appropriate spontaneous or mandatory breath type, avoiding many of the pitfalls experienced by previous ventilator Hybrid Modes that deliver only a single spontaneous breath type and only a single mandatory breath type until clinician action is taken.

Accordingly, the Adjusting Hybrid Mode as described herein may utilize several different settings, such as mandatory adjust, spontaneous adjust, and dual adjust. The Adjusting Hybrid Mode during the mandatory adjust setting automatically determines which mandatory breath type, of a plurality of mandatory breath types, should be delivered to a patient when a mandatory breath type is determined necessary by the Adjusting Hybrid Mode and delivers a selected single spontaneous breath type during a spontaneous mode. The Adjusting Hybrid Mode during a spontaneous adjust setting automatically determines which spontaneous breath type, from a plurality of spontaneous breath types, should be delivered to a patient when a spontaneous breath type is determined necessary by the Adjusting Hybrid Mode and delivers a single selected mandatory breath typed during a mandatory mode. The Adjusting Hybrid Mode during the dual adjust setting automatically determines which mandatory breath type, from a plurality of mandatory breath types, should be delivered to a patient when a mandatory breath type is determined necessary by the Adjusting Hybrid Mode and automatically determines which spontaneous breath type, from a plurality of spontaneous breath types, should be delivered to a patient when a spontaneous breath type is determined necessary by the Adjusting Hybrid Mode.

### Ventilator Breath Types

A clinician can control patient inspiration and expiration by directing a ventilator to deliver breaths of a specific breath type, usually through the selection of a mode that causes the ventilator to deliver breaths of the desired breath type. Mandatory breath types may be delivered by the ventilator or in response to a patient effort in either mandatory or mandatory/spontaneous modes of ventilation. Spontaneous breath types, on the other hand, require a spontaneously breathing patient in that the initiation is solely based on detection of a patient effort. A ventilator delivering a spontaneous breath type may trigger and/or cycle in response to a detection of patient effort. Triggering refers to the transition from expiration to inspiration in order to distinguish triggering from the transition from inspiration to expiration (referred to as cycling).

As discussed above, different patient breath types are characterized by different ventilation waveforms. In general, breath types are characterized primarily by their inhalation phase waveform and by the conditions upon which they trigger and cycle because the exhalation phase in most breaths types is a return to and holding of positive end expiratory pressure (PEEP) from the pressure at the time of cycling. The measured variables of volume, flow, pressure, and time must be calculated to produce the various waveforms.

For the purposes of the foregoing disclosure, Volume Control (VC), Pressure Control (PC), Volume-Targeted-Pressure-Control (VC+), Volume Support (VS), Pressure Support (PS), and Proportional Assist (PA) breath types will be discussed, although the reader will note that any breath type now known or later developed may be used.

### Volume-Control Breath Type

The Assist/Control (AIC) and Synchronized Intermittent Mandatory Ventilation (SIMV) modes allow a clinician to set a respiratory rate and to select a volume (from VC and VC+ breath types) to be administered to a patient during a mandatory breath. When using VC, a clinician sets a desired tidal volume, flow wave form shape, and an inspiratory flow rate or inspiratory time. These variables determine how much volume of gas is delivered to the patient and the duration of inspiration during each mandatory breath inspiratory phase. The mandatory breaths are administered according to the set respiratory rate.

For VC, when the delivered volume is equal to the prescribed tidal volume, the ventilator may initiate exhalation. Exhalation lasts from the time at which prescribed volume is reached until the start of the next ventilator mandated inspiration. This exhalation time is determined by the respiratory rate set by the clinician and any participation above the set rate by the patient. Upon the end of exhalation, another VC mandatory breath is given to the patient.

### Volume-Targeted-Pressure-Control Breath Type

The VC+ breath type is a combination of volume and pressure control breath types that may be delivered to a patient as a mandatory breath. In particular, VC+ may provide the benefits associated with setting a target tidal volume, while also allowing for variable flow. Variable flow may be helpful in meeting inspiratory flow demands for actively breathing patients.

As may be appreciated, when resistance increases it becomes more difficult to pass gases into and out of the lungs, decreasing flow. For example, when a patient is intubated, i.e., having either an endotracheal or a tracheostomy tube in place, resistance may be increased as a result of the smaller diameter of the tube over a patient's natural airway. In addition, increased resistance may be observed in patients with obstructive disorders, such as COPD, asthma, etc. Higher resistance may necessitate, inter glia, a higher inspiratory time setting for delivering a prescribed pressure or volume of gases, a lower respiratory rate resulting in a higher expiratory time for complete exhalation of gases.

Unlike VC, when the set inspiratory time is reached, the ventilator may initiate exhalation. Exhalation lasts from the end of inspiration until the beginning of the next inspiration. By controlling target tidal volume and allowing for variable flow, VC+ allows a clinician to maintain the volume while allowing the flow and pressure targets to fluctuate.

### Volume Support

Volume Support supplies a clinician-selected volume by targeting and controlling the pressure during inhalation. In the VS breath type, a clinician inputs a desired tidal volume, optionally parameters that control the change in pressure and flow between phases, and an exhalation condition such as an exhalation flow threshold. When an inhalation is triggered the ventilator calculates a target pressure from the desired tidal volume and controls to the target pressure. This target pressure is delivered until the exhalation condition is observed, at which point the ventilator cycles to PEEP. If the exhalation condition is not detected within some predetermined period of time (which may be set by the clinician), the ventilator will cycle automatically. In subsequent VS breaths, the difference between the resulting volume and the clinician-set volume is also used to calculate a revised target pressure.

### Pressure-Control Breath Type

PC allows a clinician to select a pressure to be administered to a patient during a mandatory breath. When using the PC breath type, a clinician sets a desired pressure, inspiratory time, and respiratory rate for a patient. These variables determine the pressure of the gas delivered to the patient during each mandatory breath inspiration. The mandatory breaths are administered according to the set respiratory rate.

For the PC breath type, when the inspiratory time is equal to the prescribed inspiratory time, the ventilator may initiate exhalation. Exhalation lasts from the end of inspiration until the next inspiration. Upon the end of exhalation, another PC mandatory breath is given to the patient.

During PC breaths, the ventilator may maintain the same pressure waveform at the mouth, regardless of variations in lung or airway characteristics, e.g., respiratory compliance and/or respiratory resistance. However, the volume and flow waveforms may fluctuate based on lung and airway characteristics.

### Pressure-Support Breath Type

PS is a form of assisted ventilation and is utilized in the present disclosure during a spontaneous breath. PS is a patient triggered breath and is typically used when a patient is ready to be weaned from a ventilator or for when patients are breathing spontaneously but cannot do all the work of breathing on their own. When the ventilator senses patient inspiratory effort, the ventilator provides a constant pressure during inspiration. The pressure may be set and adjusted by the clinician. The patient controls the rate, inspiratory flow, and to an extent, the inspiratory time. The ventilator delivers the set pressure and allows the flow to vary. When the machine senses a decrease in flow, or determines that inspiratory time has reached a predetermined limit, the ventilator determines that inspiration is ending. When delivered as a spontaneous breath, exhalation in PS lasts from a determination that inspiration is ending until the ventilator senses a patient effort to breathe.

### Proportional Assist

The proportional assist (PA) breath type uses automatic estimates of respiratory mechanics (lung/chest wall compliance and airway resistance) to determine the pressure to deliver to a patient. PA differs from previously discussed breath types because the ventilator provides pressure, flow, and volume proportional to patient effort. As such, PA breath type can only be used with a patient that is spontaneously triggering breaths and generating some level of inspiratory effort. The amount of pressure provided by the ventilator depends on three factors. First, the amount of pressure corresponds to the flow and volume demanded by the patient effort. Second, the amount of pressure corresponds to a degree of amplification selected by a clinician which determines the extent of ventilator response to patient effort. Third, the amount of pressure corresponds to the estimates of lung/chest wall compliance and airway resistance.

During PA, the ventilator measures the airway flow and pressure and compares these variables to the degree of amplification. When the patient triggers a breath, the ventilator delivers gas in “proportion” to these parameters based on the comparison. As a result, the greater the patient effort detected by the ventilator, the greater the amount of pressure and flow from the ventilator. An advantage of PA over previously discussed spontaneous breath types is the ability to track changes and apply support in response to patient effort.

Although the techniques introduced above and discussed in detail below may be implemented for a variety of medical devices, the present disclosure will discuss the implementation of these techniques for use in a mechanical ventilator system. The reader will understand that the technology described in the context of a ventilator system could be adapted for use with other therapeutic equipment having user interfaces, including graphical user interfaces (GUIs), for prompt startup of a therapeutic treatment.

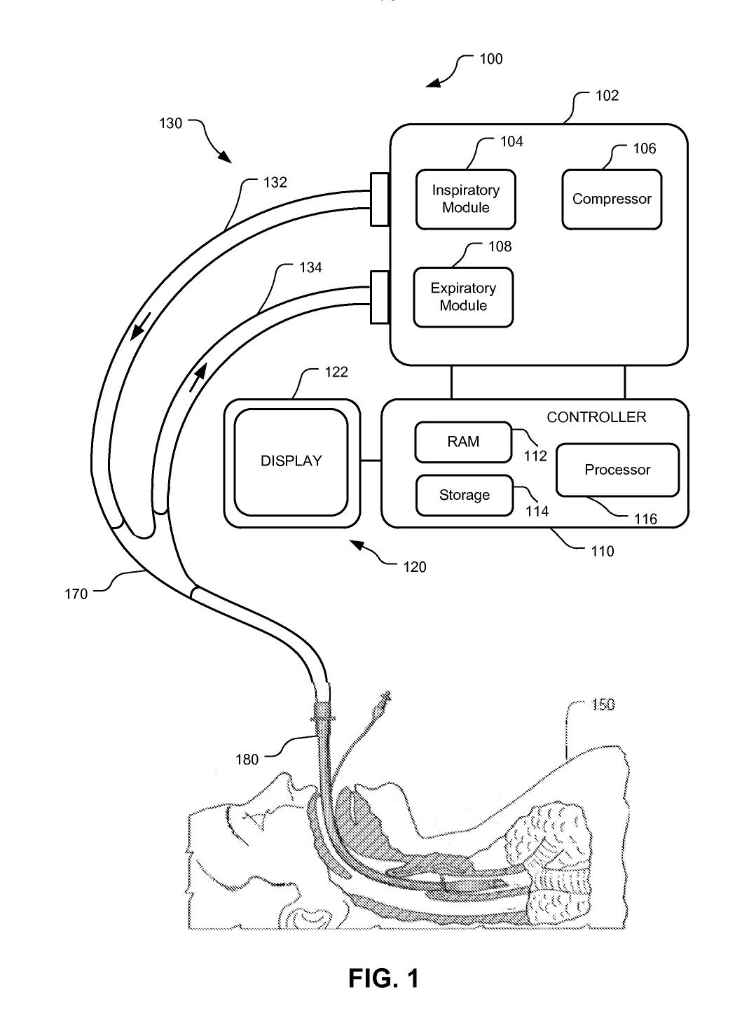

FIG. 1 is a diagram illustrating an embodiment of an exemplary ventilator 100 connected to a human patient 150. Ventilator 100 includes a pneumatic system 102 (also referred to as a pressure generating system 102) for circulating breathing gases to and from patient 150 via the ventilation tubing system 130, which couples the patient 150 to the pneumatic system 102 via an invasive (e.g., endotracheal tube, as shown) or a non-invasive (e.g., nasal mask) patient interface.

Ventilation tubing system 130 may be a two-limb (shown) or a one-limb circuit for carrying gases to and from the patient 150. In a two-limb embodiment, a fitting, typically referred to as a “wye-fitting” 170, may be provided to couple a patient interface 180 (as shown, an endotracheal tube) to an inspiratory limb 132 and an expiratory limb 134 of the ventilation tubing system 130.

Pneumatic system 102 may be configured in a variety of ways. In the present example, system 102 includes an expiratory module 108 coupled with the expiratory limb 134 and an inspiratory module 104 coupled with the inspiratory limb 132. Compressor 106 or other source(s) of pressurized gases (e.g., air, oxygen, and/or helium) is coupled with inspiratory module 104 to provide a gas source for ventilatory support via inspiratory limb 132.

The pneumatic system 102 may include a variety of other components, including mixing modules, valves, sensors, tubing, accumulators, filters, etc. Controller 110 is operatively coupled with pneumatic system 102, signal measurement and acquisition systems, and an operator interface 120 that may enable an operator or user to interact with the ventilator 100 (e.g., change ventilator settings, select operational modes, view monitored parameters, etc.). As utilized herein the term “user” and the term “operator” are interchangeable within the application. Controller 110 may include memory 112, one or more processors 116, storage 114, and/or other components of the type commonly found in command and control computing devices. In the depicted example, operator interface 120 includes a display 122 that may be touch-sensitive and/or voice-activated, enabling the display 122 to serve both as an input and output device.

The memory 112 includes non-transitory, computer-readable storage media that stores software that is executed by the processor 116 and which controls the operation of the ventilator 100. In an embodiment, the memory 112 includes one or more solid-state storage devices such as flash memory chips. In an alternative embodiment, the memory 112 may be mass storage connected to the processor 116 through a mass storage controller (not shown) and a communications bus (not shown), Although the description of computer-readable media contained herein refers to a solid-state storage, it should be appreciated by those skilled in the art that computer-readable storage media can be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Communication between components of the ventilatory system or between the ventilatory system and other therapeutic equipment and/or remote monitoring systems may be conducted over a distributed network, as described further herein, via wired or wireless means. Further, the present methods may be configured as a presentation layer built over the TCP/IP protocol. TCP/IP stands for “Transmission Control Protocol/Internet Protocol” and provides a basic communication language for many local networks (such as intra- or extranets) and is the primary communication language for the Internet. Specifically, TCP/IP is a bi-layer protocol that allows for the transmission of data over a network. The higher layer, or TCP layer, divides a message into smaller packets, which are reassembled by a receiving TCP layer into the original message. The lower layer, or IP layer, handles addressing and routing of packets so that they are properly received at a destination.

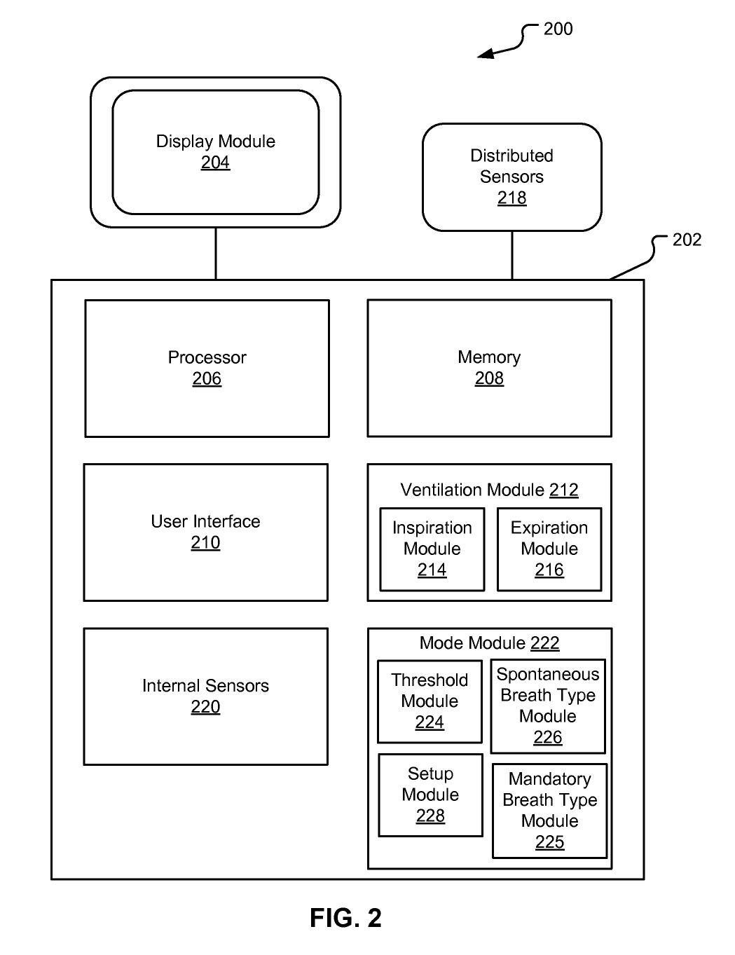

FIG. 2 is a block-diagram illustrating an embodiment of a ventilatory system for implementing at least one of an Adjusting Mandatory Mode and Adjusting Hybrid Mode of ventilation.

Ventilatory system 200 includes ventilator 202 with its various modules and components. That is, ventilator 202 may further include, inter alfa, memory 208, one or more processors 206, user interface 210, and ventilation module 212 (which may further include an inspiration module 214 and an expiration module 216). Memory 208 is defined as described above for memory 112. Similarly, the one or more processors 206 are defined as described above for one or more processors 116. Processors 206 may further be configured with a clock whereby elapsed time may be monitored by the system 200.

The ventilatory system 200 may also include a display module 204 communicatively coupled to ventilator 202. Display module 204 provides various input screens, for receiving clinician input, and various display screens, for presenting useful information to the clinician. The display module 204 is configured to communicate with user interface 210 and may include a graphical user interface (GUI). The GUI may be an interactive display, e.g., a touch-sensitive screen or otherwise, and may provide various windows and elements for receiving input and interface command operations. Alternatively, other suitable means of communication with the ventilator 202 may be provided, for instance by a wheel, keyboard, mouse, or other suitable interactive device. Thus, user interface 210 may accept commands and input through display module 204. Display module 204 may also provide useful information in the form of various ventilatory data regarding the physical condition of a patient and/or a prescribed respiratory treatment. The useful information may be derived by the ventilator 202, based on data collected by a data processing module 206, and the useful information may be displayed to the clinician in the form of graphs, wave representations, pie graphs, or other suitable forms of graphic display. For example, a settings screen may be displayed on the GUI and/or display module 204 to configure hybrid mode ventilation.

Ventilation module 212 may further include an inspiration module 214 configured to deliver gases to the patient according to prescribed ventilatory settings. Specifically, inspiration module 214 may correspond to the inspiratory module 104 or may be otherwise coupled to source(s) of pressurized gases (e.g., air, oxygen, and/or helium), and may deliver gases to the patient. Inspiration module 214 may be configured to provide ventilation according to various ventilatory breath types. As discussed above, these breath types may include PC, VC, VC+, VS, PS, and PA. Thus, the ventilation module 212 includes the algorithms and computer-readable instructions necessary to provide any desired breath type.

Ventilation module 212 may further include an expiration module 216 configured to release gases from the patient's lungs according to prescribed ventilatory settings. Specifically, expiration module 216 may correspond to expiratory module 108 or may otherwise be associated with and/or controlling an expiratory valve for releasing gases from the patient. By way of general overview, a ventilator 100 may initiate expiration based on lapse of an inspiratory time setting or other cycling criteria set by the clinician or derived from ventilator settings (e.g., detecting delivery of prescribed tidal volume or prescribed pressure). Upon initiating the expiratory phase, expiration module 216 may allow the patient to exhale by opening an expiratory valve. As such, expiration is passive, and the direction of airflow is governed by the pressure gradient between the patient's lungs (higher pressure) and the ambient surface pressure (lower pressure). Although expiratory flow is passive, it may be regulated by the ventilator based on the size of the expiratory valve opening.

According to some embodiments, the inspiration module 214 and/or the expiration module 216 may be configured to synchronize ventilation with a spontaneously-breathing, or triggering, patient. Specifically, the ventilator may detect patient effort via a pressure-monitoring method, a flow-monitoring method, direct or indirect measurement of nerve impulses, or any other suitable method. Sensing devices may be either internal 220 or distributed 218 and may include any suitable sensing device, as described further herein. In addition, the sensitivity of the ventilator to changes in pressure and/or flow may be adjusted such that the ventilator may properly detect the patient effort, i.e., the lower the pressure or flow change setting the more sensitive the ventilator may be to patient triggering. According to embodiments, a pressure-triggering method may involve the ventilator monitoring the circuit pressure, as described above, and detecting a slight drop in circuit pressure. The slight drop in circuit pressure may indicate that the patient's respiratory muscles are creating a slight negative pressure gradient between the patient's lungs and the airway opening in an effort to inspire. The ventilator may interpret the slight drop in circuit pressure as patient effort and may consequently initiate inspiration by delivering respiratory gases.

Alternatively, the ventilator may detect a flow-triggered event. Specifically, the ventilator may monitor the circuit flow, as described above. If the ventilator detects a slight drop in flow during exhalation, this may indicate, again, that the patient is attempting to inspire. In this case, the ventilator is detecting a drop in bias flow (or baseline flow) attributable to a slight redirection of gases into the patient's lungs (in response to a slightly negative pressure gradient as discussed above). Bias flow refers to a constant flow existing in the circuit during exhalation that enables the ventilator to detect expiratory flow changes and patient triggering. For example, while gases are generally flowing out of the patient's lungs during expiration, a drop in flow may occur as some gas is redirected and flows into the lungs in response to the slightly negative pressure gradient between the patient's lungs and the body's surface. Thus, when the ventilator detects a slight drop in flow below the bias flow by a predetermined threshold amount (e.g., 2 L/min below bias flow), the ventilator may interpret the drop as a patient trigger and may consequently initiate inspiration by delivering respiratory gases.

The ventilatory system 200 may also include one or more distributed sensors 218 communicatively coupled to ventilator 202. Distributed sensors 218 may communicate with various components of ventilator 202, e.g., ventilation module 212, internal sensors 220, mode module 222, threshold module 224, and any other suitable components and/or modules. Distributed sensors 218 may detect changes in patient measurements indicative of crossing a Hybrid Mode threshold, for example. Distributed sensors 218 may be placed in any suitable location, e.g., within the ventilatory circuitry or other devices communicatively coupled to the ventilator. For example, sensors may be affixed to the ventilatory tubing or may be imbedded in the tubing itself. According to some embodiments, sensors may be provided at or near the lungs (or diaphragm) for detecting a pressure in the lungs. Additionally or alternatively, sensors may be affixed or imbedded in or near wye-fitting 170 and/or patient interface 180, as described above.

Distributed sensors 218 may further include pressure transducers that may detect changes in circuit pressure (e.g., electromechanical transducers including piezoelectric, variable capacitance, or strain gauge). Distributed sensors 218 may further include various flow sensors for detecting airflow (e.g., differential pressure pneumotachometers). For example, some flow sensors may use obstructions to create a pressure decrease corresponding to the flow across the device (e.g., differential pressure pneumotachometers) and other flow sensors may use turbines such that flow may be determined based on the rate of turbine rotation (e.g., turbine flow sensors). Alternatively, sensors may utilize optical or ultrasound techniques for measuring changes in ventilatory parameters. A patient's blood parameters or concentrations of expired gases may also be monitored by sensors to detect physiological changes that may be used as indicators to study physiological effects of ventilation, wherein the results of such studies may be used for diagnostic or therapeutic purposes. Indeed, any distributed sensory device useful for monitoring changes in measurable parameters during ventilatory treatment may be employed in accordance with embodiments described herein.

Ventilator 202 may further include one or more internal sensors 220. Similar to distributed sensors 218, internal sensors 220 may communicate with various components of ventilator 202, e.g., ventilation module 212, internal sensors 220, mode module 222, threshold module 224, and any other suitable components and/or modules. Internal sensors 220 may employ any suitable sensory or derivative technique for monitoring one or more parameters associated with the ventilation of a patient. However, the one or more internal sensors 220 may be placed in any suitable internal location, such as, within the ventilatory circuitry or within components or modules of ventilator 202. For example, sensors may be coupled to the inspiratory and/or expiratory modules for detecting changes in, for example, circuit pressure and/or flow. Specifically, internal sensors 220 may include pressure transducers and flow sensors for measuring changes in circuit pressure and airflow. Additionally or alternatively, internal sensors 220 may utilize optical or ultrasound techniques for measuring changes in ventilatory parameters. For example, a patient's expired gases may be monitored by internal sensors 220 to detect physiologic changes indicative of the patient's condition and/or treatment. Indeed, internal sensors 220 may employ any suitable mechanism for monitoring parameters of interest in accordance with embodiments described herein,

As should be appreciated, ventilatory parameters are highly interrelated and, according to embodiments, may be either directly or indirectly monitored. That is, parameters may be directly monitored by one or more sensors, as described above, or may be indirectly monitored by derivation.

Ventilator system 200 may further include mode module 222. Mode module 222 is activated when a clinician indicates that the ventilator should run in a specific mode, such as SIMV Mode, A/C Mode, Spontaneous Mode, Mandatory Mode, Hybrid Mode, Adjusting Hybrid Mode, Adjusting Mandatory Mode, and Adjusting Spontaneous Mode.

Adjusting Mandatory Mode allows a ventilator to be programmed to automatically determine which mandatory breath type, of a plurality of mandatory breath types, should be delivered to a patient. During this mode, the Mandatory Breath Type Module 225 controls when and how the mandatory breath types are delivered and is in communication with the Threshold module 224. Specifically, during this mode, the Mandatory Module 225 based on its communication with the Threshold module 224 determines if the patient measurements have exceeded or fallen below a predetermined threshold associated with patient based criteria. When the Mandatory Module 225 determines that the threshold has been crossed, the Mandatory Module 225 delivers the appropriate mandatory breath type.

Adjusting Spontaneous Mode allows a ventilator to be programmed to automatically is determining which spontaneous breath type, of a plurality of spontaneous breath types, should be delivered to a patient. During this mode, the Spontaneous Breath Type Module 226 controls when and how the spontaneous breath types are delivered and is in communication with the Threshold module 224. Specifically, during this mode, the Spontaneous Module 226 based on its communication with the Threshold module 224 determines if the patient measurements have exceeded or fallen below a predetermined threshold associated with patient based criteria. When the Spontaneous Module 226 determines that the threshold has been crossed, the Spontaneous Module 226 delivers the appropriate spontaneous breath type.

Adjusting Hybrid Mode allows a ventilator to be programmed to use a first breath type in response to a spontaneous trigger (that is, when the ventilator detects that the patient is trying to inhale) and a second breath type in response to a mandatory trigger event (e.g., upon the expiration of a set time period). The Mode Module 222 in combination with the spontaneous breath type module 226 and the mandatory breath type module 225 controls when and how breath types are delivered and is in communication with the Threshold Module 224.

The Adjusting Hybrid Mode has three settings including a mandatory adjust setting, a spontaneous adjust setting, and a dual adjust setting. When a mandatory adjust setting is selected during an Adjusting Hybrid Mode, the Mode Module 222 utilizes the Spontaneous Breath Type Module 226, Mandatory Breath Type Module 225, and Threshold Module 224 to automatically determine which mandatory breath type, of a plurality of mandatory breath types, should be delivered to a patient when a patient effort is not detected within a set time period and delivers a selected single spontaneous breath type when a patient effort is detected within the set time period. The set time period may be entered or selected by the operator or determined by the ventilator based on ventilator settings and/or patient parameters. Specifically, during this setting, the Mandatory Module 225 based on its communication with the Threshold module 224 determines if the patient measurements have exceeded or fallen below a predetermined threshold associated with patient based criteria. When the Mandatory Module 225 determines that the threshold has been crossed, the Mandatory Module 225 delivers the appropriate mandatory breath type from the plurality of mandatory breath types.

When the spontaneous adjust setting is selected during an Adjusting Hybrid Mode, the Made Module 222 utilizes the Spontaneous Breath Type Module 226, Mandatory Breath Type Module 225, and Threshold Module 224 to automatically determine which spontaneous breath type, of a plurality of spontaneous breath types, should be delivered to a patient when a patient effort is detected within a set time period and delivers a single selected mandatory breath typed when a patient effort is not detected within the set time period. Specifically, during this setting, the Spontaneous Module 226 based on its communication with the Threshold module 224 determines if the patient measurements have exceeded or fallen below a predetermined threshold associated with patient based criteria. When the Spontaneous Module 226 determines that the threshold has been crossed, the Spontaneous Module 226 delivers the appropriate spontaneous breath type from the plurality of spontaneous breath types.

When the dual adjust setting is selected during an Adjusting Hybrid Mode, the Mode Module 222 utilizes the Spontaneous Breath Type Module 226, Mandatory Breath Type Module 225, and Threshold Module 224 to automatically determine which mandatory breath type, of a plurality of mandatory breath types, should be delivered to a patient when a patient effort is not detected within a set time period and automatically determine which spontaneous breath type, of a plurality of spontaneous breath types, should be delivered to a patient when a patient effort is detected within the set time period. Specifically, during this setting, the Spontaneous Module 226 based on its communication with the Threshold module 224 determines if the patient measurements have exceeded or fallen below a predetermined threshold associated with patient based criteria when a spontaneous breath type is indicated and the Mandatory Module 225 based on its communication with the Threshold module 224 determines if the patient measurements have exceeded or fallen below a predetermined threshold associated with patient based criteria when a mandatory breath type is indicated. When the Spontaneous Module 226 or Mandatory Module 225 determines that the threshold has been crossed, the Spontaneous Module 226 or the Mandatory Module 225 delivers the appropriate breath type.

The Mode module 222 is communicatively coupled to the threshold module 224, the spontaneous breath type module 226, and the mandatory breath type module 225. In some embodiments, threshold module 224 is configured to detect when patient measurements have crossed a predetermined threshold indicative of a patient's effort to initiate a breath. The predetermined threshold serves as an indicator that the Mode module 222 should deliver the breath type selected for spontaneous breathing. For example, a tidal volume threshold may be set for 80%, an inspiratory pressure threshold may be set for 12 cm H₂O, and a rapid shallow breathing index threshold may be set to 100. As will be appreciated, these are some of many thresholds that may be crossed, all of which are within the scope of the present disclosure. When a threshold is exceeded, the threshold module 224 communicates an exceeded threshold to the spontaneous breath type module 226.

The Mode module 222 is also communicatively coupled to the spontaneous breath type module 226. Upon indication that the ventilator should deliver a spontaneous breath type, the spontaneous breath type module 226 communicates to the ventilator an appropriate spontaneous breath type for delivery.

During a spontaneous adjust or dual adjust setting, the spontaneous breath type module 226 determines which spontaneous breath type is appropriate for the patient through communication with the threshold module 224. For example, the threshold module 224 may communicate to the spontaneous breath type module 226 that a threshold has been crossed. The spontaneous breath type module 226 may then process this information to determine an appropriate spontaneous breath type. For example, if the tidal volume is less than 80% of a set threshold, the spontaneous breath type module 226 may communicate to the ventilator that the spontaneous breath type should be VS instead of PA. If the inspiratory pressure drops below 12 cm H₂O, the spontaneous breath type module 226 may indicate to the ventilator that PA should be used instead of VS. If the rapid shallow breathing index is greater than 100, the spontaneous breath type module 226 may communicate to the ventilator that VS should be used as the spontaneous breath type instead of PA. As will be appreciated, these thresholds are exemplary and many different thresholds are contemplated within the scope of the present disclosure. Determining an appropriate spontaneous breath type will be discussed in further detail below.

During a mandatory adjust setting of the Adjusting Hybrid Mode, the spontaneous breath type module 226 delivers a single selected spontaneous breath type to the patient when a patient effort is detected by the ventilator within the set time period by the threshold module 224. During this setting, only a single spontaneous breath type is selected and delivered to the patient. The spontaneous breath type is not changed by the ventilator unless the selected spontaneous breath type is manually changed by the clinician. The selected spontaneous breath type may be determined by the ventilator and/or may be input or selected by the clinician.

During a mandatory adjust or dual adjust setting, the mandatory breath type module 225 determines which mandatory breath type is appropriate for the patient through communication with the threshold module 224. For example, the threshold module 224 may communicate to the mandatory breath type module 225 that a patient effort was not detected within the set time period and that a threshold has been crossed. The mandatory breath type module 225 may then process this information to determine an appropriate mandatory breath type.

For example, if the lung/chest wall compliance decreases below a predetermined amount or by a predetermined amount, the mandatory breath type module 225 may communicate to the ventilator that the mandatory breath type should be VC+ or PC instead of VC. If the lung/chest wall compliance increases above a predetermined amount or by a predetermined amount, the mandatory breath type module 225 may communicate to the ventilator that the mandatory breath type should be VC+ or VC instead of PC. If the tidal volume increases or decreases above and/or below a predetermined threshold, the mandatory breath type module 225 may communicate to the ventilator that VC or VC+ should be used as the mandatory breath type instead of PC. In another example, if partial pressure of carbon dioxide in the arterial blood (PaCO₂) increases or decreases above and/or below a predetermined threshold, the mandatory breath type module 225 may communicate to the ventilator that VC or VC+ should be used as the mandatory breath type instead of PC. In a further example, if the airway resistance or peak inspiratory pressure increases above a predetermined threshold or by a predetermined amount, the mandatory breath type module 225 may communicate to the ventilator that the mandatory breath type should be VC+or PC instead of VC. In another example, if the oxygen saturation level of the blood (SpO₂) decreases below a predetermined threshold or by a predetermined amount, the mandatory breath type module 225 may communicate to the ventilator that the mandatory breath type should be VC+ or PC instead of VC. As will be appreciated, these thresholds are exemplary and many different thresholds are contemplated within the scope of the present disclosure. Determining an appropriate mandatory breath type will be discussed in further detail below.

Accordingly, the Mode module 222 is also communicatively coupled to setup module 228. Setup module 228 is coupled with display module 204 to provide configuration options for an Adjusting Mandatory Mode and/or for an Adjusting Hybrid Mode at setup. Specifically, setup module 228 provides display module 204 with two configuration options for the Adjusting Mandatory Mode and/or the Adjusting Hybrid Mode. The first configuration option is “Easy Mode” and configures the ventilator to operate in Adjusting Mandatory Mode using preselected mandatory breath types. In one embodiment, “Easy Mode” automatically designates VC, PC and VC+ as the mandatory breath types. The first configuration option “Easy Mode” configures the ventilator to operate in the Adjusting Hybrid Mode using preselected mandatory and spontaneous breath types. In one embodiment, “Easy Mode” automatically designates PA and VS as the spontaneous breath types and PC, VC, and VC+ as the mandatory breath types.

The second configuration option provided by the setup module 228 is “Config Mode.” The “Config Mode” is intended for the more sophisticated operatory or user that wants maximum control over the ventilator. When the setup module 228 receives an indication that the clinician has chosen “Config Mode,” it provides a list of all available mandatory breath types for selection by the clinician in the Adjusting Mandatory mode. When the setup module 228 receives an indication that the clinician has chosen “Config Mode,” it provides a list of all available spontaneous breath types and mandatory breath types for selection by the clinician during the Adjusting Hybrid mode. The clinician may then select one or more spontaneous breath types and/or mandatory breath types for delivery to the patient. The setup module 228 communicates the selected breath types to the threshold module 224 and mode module 222.

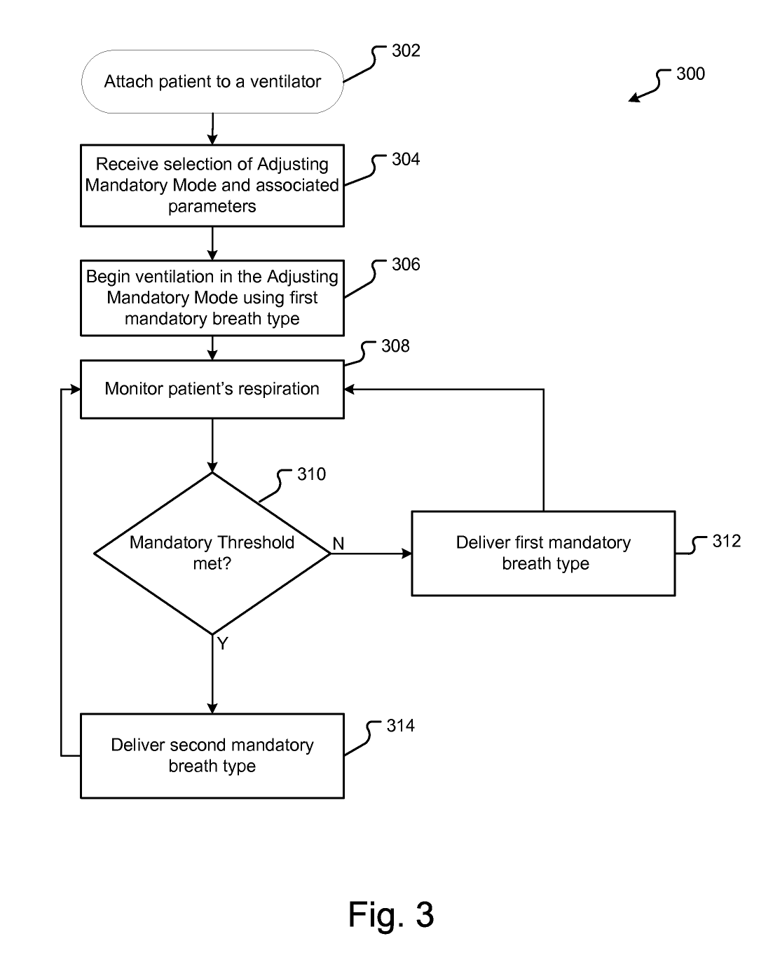

FIG. 3 represents an embodiment of a method 300 for operating a ventilator in an Adjusting Mandatory Mode. The Adjusting Mandatory Mode automatically determines which mandatory breath type, of a plurality of mandatory breath types, should be delivered to a patient. As illustrated, method 300 includes an attach operation 302. At attach operation 302, a patient is attached to a ventilator.

Method 300 includes a receive operation 304. At receive operation 304, an indication is received that the ventilator is set to operate in Adjusting Mandatory Mode.

Such an indication may come from a graphical user interface that displays “Adjusting Mandatory Mode” as a selectable element. In other embodiments, the indication is received from a determination by the ventilator based on measured patient parameters and ventilator settings.

The indication that the ventilator is set to operate in Adjusting Mandatory Mode is accompanied by the breath type parameters to be used during mandatory breaths. In one embodiment, the breath type parameters are preselected as the clinician has chosen to setup Adjusting Mandatory Mode using an “Easy Mode.” For example, setting up Adjusting Mandatory Mode with “Easy Mode” may communicate that VC, PC and VC+ mandatory breath types should be used. Alternatively, the breath type parameters are designated by a clinician using a “Config Mode.” If the clinician sets up Adjusting mandatory Mode using “Config Mode,” any available mandatory breath type(s) may be selected. For the purposes of this discussion, VC, PC, and VC+ will be described as the selected mandatory breath types. However, it will be appreciated that any mandatory breath types may be utilized for the purposes of the present application. The mandatory breath types are communicated alongside the indication that the ventilator is set to operate in Adjusting Mandatory Mode.

Further, method 300 includes a begin operation 306. At begin operation 306, the ventilator begins ventilation in Mandatory Adjusting Mode by delivering a first mandatory breath type. In one embodiment, the first mandatory breath type is VC. In one embodiment, the first mandatory breath type is PC. The first mandatory breath type may be selected or derived by the ventilator based on patient parameters and/or ventilator settings or may input or selected by an operator.

As illustrated, method 300 includes a monitor operation 308. At the monitor operation 308, the ventilator monitors patient based criteria. The patient measurements may be monitored per breath, after a predetermined time period, after exhalation, after inhalation, and/or after a predetermined number of breaths. The monitoring is done by any of the internal and/or distributed sensors discussed above. The sensors can measure any relevant patient based criteria including but not limited to work of breathing, patient effort, carbon dioxide, inspiratory pressure, expiratory pressure, respiratory rate, inspiratory volume, expiratory volume, body weight, minute ventilation, lung/chest wall compliance, tidal volume, airway resistance, PaCO₂, SpO₂, peak inspiratory pressure, and target pressure.

These patient based criteria are used by the ventilator to determine whether the patient is being administered the appropriate breath type.

Further, method 300 includes a detect operation 310. At detect operation 310, a determination is made as to whether a threshold associated with the patient based criteria has been crossed. For example, a determination may be made as to whether a patient's lung/chest wall compliance is below a predetermined threshold. If the patient's lung/chest wall compliance is below the predetermined threshold, then a determination may be made that the appropriate mandatory breath type is not being delivered and the ventilator selects to perform second deliver operation 314. If the patient's lung/chest wall compliance is equal to or above the predetermined threshold, then a determination may be made that the appropriate mandatory breath type is being delivered and the ventilator selects to perform first deliver operation 312.

For example, the ventilator may be currently delivering the patient a PC mandatory breath type, but the patient has a tidal volume below a predetermined threshold. As a result, the patient is not receiving enough tidal volume and the PC mandatory breath type may no longer be appropriate. If a determination is made that the appropriate mandatory breath type is not being delivered, the ventilator selects to perform second deliver operation 314. In this example, the ventilator may select to deliver a VC or VC+ breath during the second deliver operation 314 to provide the appropriate tidal volume to the patient. It will be appreciated by a person of skill in the art that other patient parameters may be utilized to determine if the appropriate mandatory breath type is being delivered to the patient, such as but not limited to work of breathing, patient effort, carbon dioxide, inspiratory pressure, expiratory pressure, respiratory rate, inspiratory volume, expiratory volume, body weight, minute ventilation, lung/chest wall compliance, tidal volume, airway resistance, PaCO₂, SpO₂, peak inspiratory pressure, and target pressure.

Additionally, method 300 includes a first deliver operation 312. At first deliver operation 312, the first mandatory breath type is delivered to the patient. For example, the patient may be delivered a PC, VC, or VC+ mandatory breath type. As discussed above the first mandatory breath type may be determined or derived by the ventilator based on ventilator settings and/or monitored patient criteria or may be input or selected by an operator.

As illustrated, method 300 includes a second deliver operation 314. At second deliver operation 314, a second mandatory breath type different from the first mandatory breath type is delivered to the patient. For example, the patient may be delivered a PC, VC, or VC+ mandatory breath type. The second mandatory breath type delivered to the patient is based on the determination made by the detect operation 310. For example, if the ventilator determines during the threshold operation 310 when a PC breath type is being delivered to a patient that the patient's lung/chest wall compliance has increased by a predetermined threshold amount, the ventilator will select to perform second deliver operation 314 to deliver a different mandatory breath type, such as VC or VC+ to the patient.

Embodiments of method 300 may utilize different mandatory breath types other than VC, PC, and VC+. Moreover, any number of mandatory breath types may be administered in combination within the scope of the present disclosure. For example, a clinician may select more than two or three mandatory breath types.

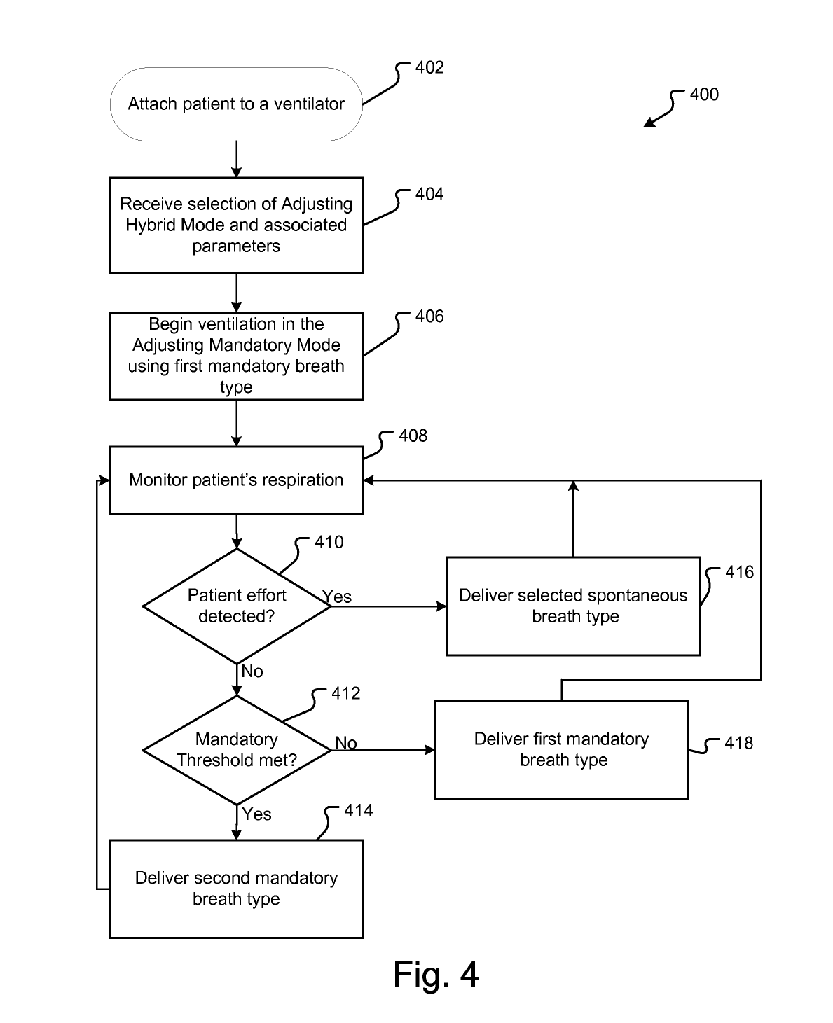

FIG. 4 represents an embodiment illustrating a method 400 for operating a ventilator in Adjusting Hybrid Mode in a mandatory adjust setting. As illustrated, method 400 includes an attach operation 402. At attach operation 402, a patient is attached to a ventilator.

Method 400 includes a receive operation 404. At receive operation 404, an indication is received that the ventilator is set to operate in Adjusting Hybrid Mode in a mandatory adjust setting. Such an indication may come from a graphical user interface that displays “Adjusting Hybrid Mode” as a selectable element. The indication that the ventilator is set to operate in Adjusting Hybrid Mode in a mandatory adjust setting is accompanied by the breath type parameters to be used during mandatory breaths and the breath type parameter to be used during the selected spontaneous breath type. In one embodiment, the breath type parameters are preselected as the clinician has chosen to setup

Adjusting Hybrid Mode using an “Easy Mode.” For example, setting up Adjusting Hybrid Mode in a mandatory adjust setting with “Easy Mode” may communicate that PA is the spontaneous breath type and VC, PC, and VC+ are the mandatory breath types to be used. Alternatively, the breath type parameters are designated by a clinician using a “Config Mode.” If the clinician sets up Adjusting Hybrid Mode using “Config Mode,” any available spontaneous breath type and mandatory breath type(s) may be selected. For the purposes of this discussion, PA will be described as the selected spontaneous breath type and VC, PC, and VC+ will be described as the selected mandatory breath types. However, it will be appreciated that any spontaneous breath type and any mandatory breath types may be utilized for the purposes of the present application.

At begin operation 406, the ventilator begins ventilation in Adjusting Hybrid Mode by delivering a first mandatory type. In one embodiment, the first mandatory breath type is VC. The first mandatory breath type delivered by the ventilator may be determined by the ventilator based on monitored patient criteria or may be input or selected by an operator. In another embodiment, the “Easy Mode” may automatically designate the first mandatory breath type delivered to the patient as VC, VC+ or PC.

Method 400 includes a monitor operation 408. At the monitor operation 408, the ventilator monitors patient based criteria. Adjusting Hybrid Mode operates on a breath to breath basis. As a result, patient measurements are monitored per breath. The monitoring is done by any of the internal and/or distributed sensors discussed above. The sensors can measure any relevant patient based criteria including but not limited to work of breathing, patient effort, carbon dioxide, inspiratory pressure, expiratory pressure, respiratory rate, inspiratory volume, expiratory volume, body weight, minute ventilation, lung/chest wall compliance, tidal volume, airway resistance, PaCO₂, SpO₂, peak inspiratory pressure, and target pressure. These patient based criteria are used by the ventilator to determine whether the patient is being administered the appropriate breath type. In one embodiment, the sensors can only detect patient effort during exhalation. In another embodiment, the sensors can detect patient effort during both inspiration and exhalation.

Further, method 400 includes detect operation 410. At detect operation 410, a determination is made as to whether a patient inspiratory effort is detected. If a patient inspiratory effort is detected within a set time period (or backup rate) by the ventilator during detect operation 410, the ventilator selects to perform spontaneous deliver operation 416. The set time period may be entered or selected by the operator or determined by the ventilator based on ventilator settings and/or patient parameters. If a patient inspiratory effort is not detected within the set time period (or backup rate) by the ventilator during is detect operation 410, the ventilator selects to perform threshold operation 412. The ventilator may detect patient inspiratory effort via a pressure-monitoring method, a flow-monitoring method, direct or indirect measurement of nerve impulses, or any other suitable method.

Method 400 includes spontaneous deliver operation 416. At spontaneous deliver operation 416 a selected spontaneous breath type is delivered to the patient. The selected spontaneous breath type, such as PA, is delivered to the patient unless a clinician changes the selected spontaneous breath type. In some embodiments, the selected spontaneous breath type may be input or selected by a clinician. In other embodiments, the selected spontaneous breath type is determined by the ventilator based on ventilator settings and/or the monitored patient based criteria.

As illustrated, method 400 includes a threshold operation 412. At threshold operation 412, a determination is made as to whether a threshold associated with the patient based criteria has been crossed. For example, a determination may be made as to whether a patient's lung/chest wall compliance is below a predetermined threshold. If the patient's lung/chest wall compliance is below the predetermined threshold, then a determination may be made that the appropriate mandatory breath type is not being delivered and the ventilator selects to perform second mandatory deliver operation 414. If the patient's lung/chest wall compliance is equal to or above the predetermined threshold, then a determination may be made that the appropriate mandatory breath type is being delivered and the ventilator selects to perform first mandatory deliver operation 418.

For example, if the first mandatory breath type during the begin operation 406 or first mandatory deliver operation 418 being delivered to the patient is a VC breath type and the ventilator during the threshold operation 412 determines that the peak inspiratory pressure is above a peak inspiratory pressure threshold, the ventilator selects to deliver VC+or PC to the patient during the second mandatory deliver operation 414. However, if the ventilator during the threshold operation 412 determines that the peak inspiratory pressure is not above a peak inspiratory pressure threshold, the ventilator selects to deliver VC to the patient during the first mandatory deliver operation 418. Additionally, method 400 includes a first mandatory deliver operation 418. At first mandatory deliver operation 418, the first mandatory breath type is delivered to the patient. For example, the patient may be delivered a PC, VC, or VC+ mandatory breath type. As discussed above, the first mandatory breath type may be determined by the ventilator based on ventilator settings and/or the monitored patient based criteria or may be input and/or selected by an operator.

As illustrated, method 400 includes a second mandatory deliver operation 414. At second mandatory deliver operation 414, a second mandatory breath type different from the first mandatory breath type is delivered to the patient. For example, the patient may be delivered a PC, VC, or VC+ mandatory breath type. The second mandatory breath type delivered to the patient is based the determination made by the threshold operation 412. For example, if the ventilator determines during the threshold operation 412 that the PaCO₂ of the patient has increased or decreased by a predetermined amount, the ventilator may select to switch from the PC breath type to a VC or VC+ breath type during the second mandatory deliver operation 414.

Embodiments of the method 400 may utilize different spontaneous breath types than PA and VS and different mandatory breath types than VC, PC, and VC+. Moreover, any number of mandatory breath types may be administered in combination within the scope of the present disclosure. For example, a clinician may select more than two mandatory breath types.

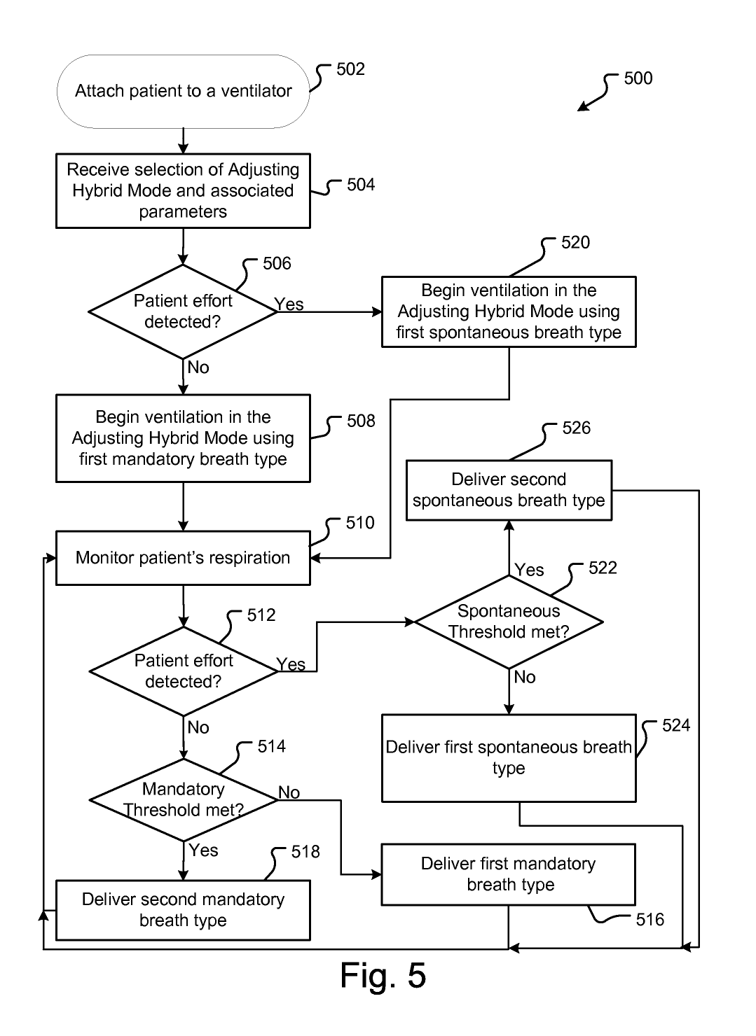

FIG. 5 represents an embodiment illustrating a method 500 for operating a ventilator in Adjusting Hybrid Mode in a dual setting. Method 500 includes an attach operation 502. At attach operation 502, a patient is attached to a ventilator.

As illustrated, method 500 includes a receive operation 504. At receive operation 504, an indication is received that the ventilator is set to operate in Adjusting Hybrid Mode in a dual adjust setting. Such an indication may come from a graphical user interface that displays “Adjusting Hybrid Mode” as a selectable element. The indication that the ventilator is set to operate in Adjusting Hybrid Mode in a dual adjust setting is accompanied by the breath type parameters to be used during spontaneous breaths and mandatory breaths. In one embodiment, the breath type parameters are preselected as the clinician has chosen to setup Adjusting Hybrid Mode in a dual adjust setting using an “Easy Mode.” For example, setting up Adjusting Hybrid Mode in a dual adjust setting with “Easy Mode” may communicate that PA and VS spontaneous breath types should be used and VC, PC, and VC+ breath types should be used. Alternatively, the breath type parameters are designated by a clinician using a “Config Mode.” If the clinician sets up Adjusting Hybrid Mode in a dual adjust setting using “Config Mode,” any available spontaneous and mandatory breath type(s) may be selected. For the purposes of this discussion, PA and VS will be described as the selected spontaneous breath types and PC, VC, and VC+ will be described as the selected mandatory breath types. However, it will be appreciated that any spontaneous or mandatory breath types may be utilized for the purposes of the present application. The spontaneous and mandatory breath types are communicated alongside the indication that the ventilator is set to operate in Adjusting Hybrid Mode in a dual adjust setting.

Further, method 500 includes first detect operation 506. At first detect operation 506, a determination is made as to whether a patient inspiratory effort is detected within a set time period (or backup rate). The set time period may be entered or selected by the operator or determined by the ventilator based on ventilator settings and/or patient parameters. If a patient inspiratory effort is detected within the set time period (or backup rate) by the ventilator during first detect operation 506, the ventilator selects to perform spontaneous begin operation 520. If a patient inspiratory effort is not detected within the set time period (or backup rate) by the ventilator during first detect operation 506, the ventilator selects to perform mandatory begin operation 508. The ventilator may detect patient inspiratory effort via a pressure-monitoring method, a flow-monitoring method, direct or indirect measurement of nerve impulses, or any other suitable method.

Method 500 includes a mandatory begin operation 508. At mandatory begin operation 508, the ventilator begins ventilation in Adjusting Hybrid Mode by delivering a first mandatory breath type. In one embodiment, the first mandatory breath type is VC. In another embodiment, the first mandatory breath type is PC.

Further, method 500 includes a spontaneous begin operation 520. At spontaneous begin operation 520, the ventilator begins ventilation in Adjusting Hybrid Mode in a dual adjust setting by delivering a first spontaneous breath type. In one embodiment, the first spontaneous breath type is PA.

As illustrated, method 500 includes a monitor operation 510. At the monitor operation 510, the ventilator monitors patient based criteria, Adjusting Hybrid Mode in a dual adjust setting operates on a breath to breath basis. As a result, patient measurements are monitored per breath. The monitoring is done by any of the internal and/or distributed sensors discussed above. The sensors can measure any relevant patient based criteria including but not limited to work of breathing, patient effort, carbon dioxide, inspiratory pressure, expiratory pressure, respiratory rate, inspiratory volume, expiratory volume, body weight, minute ventilation, lung/chest wall compliance, tidal volume, airway resistance, PaCO₂, SpO₂, peak inspiratory pressure, and target pressure. These patient based criteria are used by the ventilator to determine whether the patient is being administered the appropriate breath type. In one embodiment, the sensors can only detect patient effort during exhalation. In another embodiment, the sensors can detect patient effort during both inspiration and exhalation.

Method 500 includes a second detect operation 512. At second detect operation 512 a determination is made as to whether a patient inspiratory effort is detected within the set time period (or backup rate). If a patient inspiratory effort is detected within the set time period (or backup rate) by the ventilator during second detect operation 512, the ventilator selects to perform spontaneous threshold operation 522. If a patient inspiratory effort is not detected within the set time period (or backup rate) by the ventilator during second detect operation 512, the ventilator selects to perform mandatory threshold operation 514. As discussed above, the ventilator may detect patient inspiratory effort via a pressure-monitoring method, a flow-monitoring method, direct or indirect measurement of nerve impulses, or any other suitable method.

As illustrated, method 500 includes a mandatory threshold operation 514. At mandatory threshold operation 514, a determination is made as to whether a threshold associated with the patient based criteria has been crossed. For example, a determination may be made as to whether a patient's SpO₂ is below a predetermined threshold during the delivery of a VC breath type. If the patient's SpO₂ is below the predetermined threshold, then a determination may be made that the appropriate mandatory breath type is not being delivered and the ventilator selects to perform second mandatory deliver operation 518. If the patient's SpO₂ is equal to or above the predetermined threshold during the delivery of a VC breath type, then a determination may be made that the appropriate mandatory breath type is being delivered and the ventilator selects to perform first mandatory deliver operation 516.

Additionally, method 500 includes a first mandatory deliver operation 516. At first mandatory deliver operation 516 the first mandatory breath type is delivered to the patient.

For example, the patient may be delivered a PC, VC, or VC+ mandatory breath type. The first mandatory breath type may be determined or derived by the ventilator based on monitored patient criteria and/or ventilator settings or may be input or selected by the operator.

As illustrated, method 500 includes a second mandatory deliver operation 518. At second mandatory deliver operation 518, a second mandatory breath type different from the first mandatory breath type is delivered to the patient. For example, the patient may be delivered a PC, VC, or VC+ mandatory breath type. The second mandatory breath type delivered to the patient is based on the determination made by the mandatory threshold operation 514. For example, if during the delivery of the first mandatory breath type of PC, the ventilator during the mandatory threshold operation 514 determines that the tidal volume is above or below a predetermined tidal volume threshold, the ventilator selects to deliver a VC or VC+ breath type during second mandatory deliver operation 518. If the ventilator determines that the tidal volume is within a predetermined tidal volume threshold, the ventilator selects to deliver the PC breath type during first mandatory deliver operation 516.

Additionally, method 500 includes a spontaneous threshold operation 522. At spontaneous threshold operation 522, a determination is made as to whether a threshold associated with the patient based criteria has been crossed. For example, a determination may be made as to whether a patient is displaying an effort that is too weak, If the patient effort is not too weak, then a determination may be made that the appropriate spontaneous breath type is being delivered. Accordingly, the ventilator during the spontaneous threshold operation 522 selects to perform first spontaneous deliver operation 524. However, the ventilator may be currently delivering the patient PA spontaneous breath type but the patient is displaying weak effort. As a result the patient is not receiving enough volume and the PA spontaneous breath type may no longer be appropriate. If a determination is made by the ventilator during the spontaneous threshold operation 522 that the appropriate spontaneous breath type is not being delivered, the ventilator selects to perform second spontaneous deliver operation 526. Method 500 includes a first spontaneous deliver operation 524. At first spontaneous deliver operation 524, the first spontaneous breath type is delivered to the patient. For example, the patient may be delivered a VS or PA spontaneous breath type. By delivering the patient a VS spontaneous breath type, the patient will be delivered a set volume, helping the patient who is exhibiting weak inspiratory effort.

Method 500 includes a second spontaneous deliver operation 526. At second spontaneous deliver operation 526, a second spontaneous breath type is delivered to the patient. The second spontaneous breath type is different from the first spontaneous breath type. For example, the patient may be delivered a VS spontaneous breath type if the first spontaneous breath type was PA. By delivering the patient a VS spontaneous breath type, the patient will be delivered a set volume, helping the patient who is exhibiting weak inspiratory effort. The second spontaneous breath type delivered to the patient is based on the determination made by the spontaneous threshold operation 522.

Embodiments of the method 500 may utilize different spontaneous breath types than PA and VS and different mandatory breath types than VC, PC, and VC+. Moreover, any number of spontaneous and/or mandatory breath types may be administered in combination within the scope of the present disclosure. For example, a clinician may select more than two spontaneous breath types and more than three mandatory breath types.

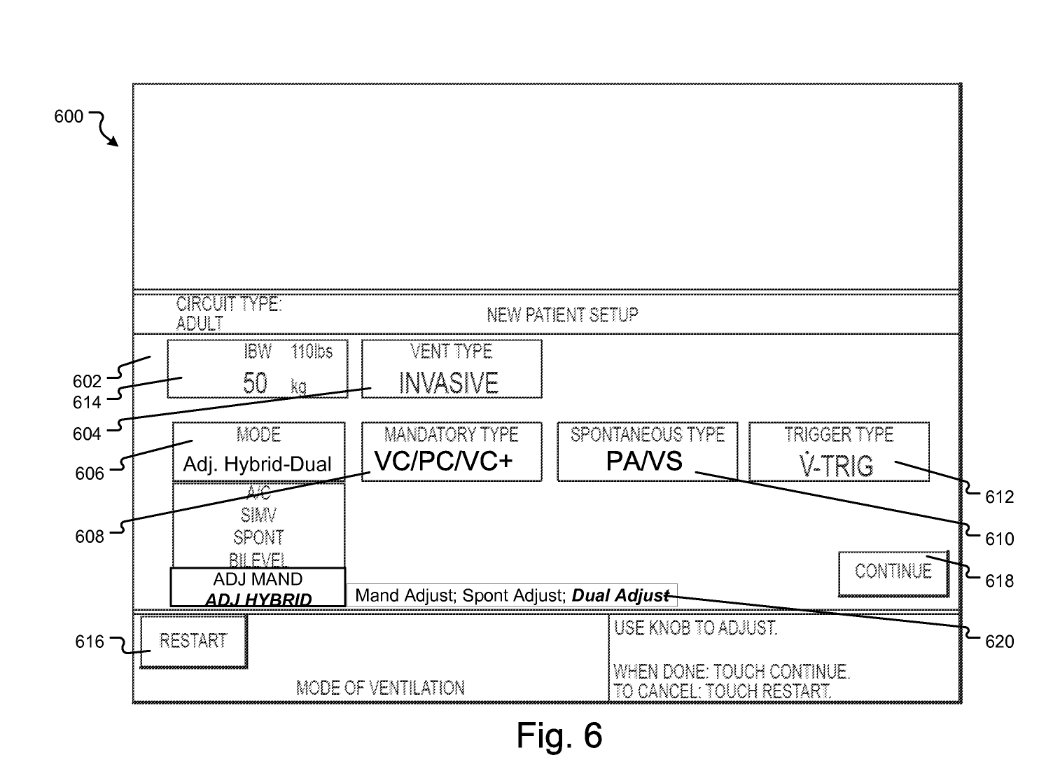

FIG. 6 is an illustration of a user interface for setting up a new patient attached for ventilation using Adjusting Hybrid Mode and Adjusting Mandatory Mode.

For the purposes of the foregoing discussion, the user interfaces may be accessed via any suitable means, for example via a main ventilatory user interface on display module. As illustrated, the user interfaces may provide one or more windows for display and one or more elements for selection and/or input. Windows may include one or more elements and, additionally, may provide graphical displays, instructions, or other useful information to the clinician. Elements may be displayed as buttons, tabs, icons, toggles, or any other suitable visual access element, etc., including any suitable element for input selection or control.

According to one embodiment, as illustrated by FIG. 6, new patient setup interface 600 may include new patient setup window 602. New patient setup window 602 may include one or more selectable elements to configure new patient setup. Accordingly, the new patient setup interface may accept commands entered by the operator through the graphical user interface. New patient setup window 602 may include a Vent Type button 604. Vent Type button 604 allows a clinician to select a type of ventilation for the patient. In one embodiment, when the clinician selects the Vent Type button 604 a pull down menu appears underneath the Vent Type button 604 displaying vent type options (not depicted). The clinician can then select one of the vent type options to set as the Vent Type. The vent type options may include invasive and non-invasive. These vent type options correspond to the way that the patient was attached to the ventilator as discussed in detail with reference to FIG. 1. As will be appreciated, when a vent type option is selected, it is displayed in the Vent Type button 604 as depicted in FIG. 6.

New patient setup window 602 may be further configured to include a Mode button 606. Like the Vent Type button 604, when a clinician selects the Mode button 606, a pull down menu appears under the Mode button 606. The pull down menu displays various modes options for selection. In one embodiment, the pull down menu includes an Adjusting Mandatory Mode (shown as ADJ MAND) and an Adjusting Hybrid Mode (shown as ADJ HYBRID) option. In another embodiment, the pull down menu includes Hybrid-Easy and Hybrid-Config options (not illustrated). As discussed above, the Hybrid-Easy option may be selected for a preconfigured Adjusting Mandatory Mode or Adjusting Hybrid Mode ventilator setup. The Hybrid-Config option may be selected by an operator or user who wants to specify each spontaneous breath type and/or mandatory breath type utilized during Adjusting Mandatory Mode and/or the Adjusting Hybrid Mode. As will be appreciated, when a mode option is selected, it is displayed in the Mode button 606 as depicted in FIG. 6.

In some embodiments, when the Adjusting Hybrid Mode is selected in the drop down menu of the Mode button 606, a second drop down window or setting window 620 appears listing the settings of the Adjusting Hybrid Mode for selection, such as Mandatory Adjust (shown as Mand Adjust), Spontaneous Adjust (shown as Spont Adjust), and Dual Adjust. The setting window 620 allows a clinician to select the setting of the Adjusting Hybrid Mode after selecting to utilize the Adjusting Hybrid Mode.

The new patient setup window 602 may be further configured to include a Mandatory Type button 608. When the clinician selects the Mandatory Type button 608 a pull down menu appears under the Mandatory Type button 608. The pull down menu displays various mandatory breath type options for selection, such as VC, PC, and VC+. Accordingly, the mandatory type options are mandatory breath types. Depending upon the mode setting the clinician may select one or more mandatory breath types from the

Mandatory Type button 608. As will be appreciated, when a mandatory breath type option is selected, it is displayed in the Mandatory Type button 608 as depicted in FIG. 6. In one embodiment, if the Mode button 606 is set to Adj-Hybrid-Mand-Easy, the Mandatory Type button 608 is automatically set to PC, VC, and VC+. In another embodiment, if the Mode button 606 is set to Adj-Hybrid-Mand-Config, the clinician can choose from any of the mandatory breath types listed under the Mandatory Type button 608.

The new patient setup window 602 may be further configured to include a Spontaneous Type button 610. When the clinician selects the Spontaneous Type button 610 a pull down menu appears under the Spontaneous Type button 610. The pull down menu displays at least various spontaneous breath type options for selection, such as VS and PA.

Depending upon the modes setting, the clinician may select one or more spontaneous breath types from the Spontaneous Type button 610. As will be appreciated, when a spontaneous breath type is selected, it is displayed in the Spontaneous Type button 610 as depicted in FIG. 6.

In one embodiment, if the Mode button 606 is set to Adj-Hybrid-Dual-Easy or Adj-Spont-Easy, the Spontaneous Type button 610 is automatically set to PA or VS. In another embodiment, if the Mode button 606 is set to Adj-Hybrid-Dual-Config or Adj-Spont-Easy, the clinician can choose from any of the spontaneous breath types.

The new patient setup window 602 may be further configured to include a Trigger Type button 612. When the clinician selects the Trigger Type button 612 a pull down menu appears under the Trigger Type button 612. The pull down menu displays various trigger type options for selection. These trigger types may include a flow trigger and a pressure trigger. As will be appreciated, the selected trigger type determines the patient measurement(s) used to determine if a patient is spontaneously triggering. In one embodiment, if the Mode button 606 is set to Adj-Hybrid-Dual-Easy, the Trigger Type button 612 is automatically set to flow trigger. In another embodiment, if the Mode button 606 is set to Adj-Hybrid-Dual-Config, the clinician can choose from any available trigger types such as pressure, flow, volume, patient effort, etc. As will be appreciated, when a trigger type option is selected, it is displayed in the Trigger Type button 612 as depicted in FIG. 6.

The new patient setup window 602 may include various other selectable elements. For example, the window may include an Ideal Body Weight button 614 and a restart button 616, Like the other buttons discussed above with reference to FIG. 6, the Ideal Body Weight button 614 may be selected to change the Ideal Body Weight setting of a patient. The restart button 616 may also be selected to restart the ventilator.

Once a clinician is satisfied with the settings displayed on the new patient setup window 602, the clinician may select the continue button 618 to configure the ventilator with the displayed settings. When the continue button 618 has been selected, the ventilator may display a ventilator settings interface.

It will be clear that the systems and methods described herein are well adapted to attain the ends and advantages mentioned as well as those inherent therein. Those skilled in the art will recognize that the methods and systems within this specification may be implemented in many manners and as such is not to be limited by the foregoing exemplified embodiments and examples. In other words, functional elements being performed by a single or multiple components, in various combinations of hardware and software, and individual functions can be distributed among software applications at either the client or server level. In this regard, any number of the features of the different embodiments described herein may be combined into one single embodiment and alternative embodiments having fewer than or more than all of the features herein described are possible.

While various embodiments have been described for purposes of this disclosure, various changes and modifications may be made which are well within the scope of the present technology. Numerous other changes may be made which will readily suggest themselves to those skilled in the art and which are encompassed in the spirit of the disclosure and as defined in the appended claims.

What is claimed is:

1. A method for operating a ventilator, the method comprising:
receiving a user selection of two or more mandatory breath types from a plurality of mandatory breath types;
monitoring one or more patient respiratory parameters during ventilation of a patient;
comparing at least one monitored patient respiratory parameter to at least one predetermined mandatory threshold; and
delivering a mandatory breath of a selected one of the two or more selected mandatory breath types to the patient based on results of the mandatory comparing operation.

2. The method of claim 1, wherein the ventilator is operating in an Adjusting Hybrid Mode.

3. The method of claim 2, further comprising delivering a spontaneous breath of a preselected spontaneous breath type when a patient effort is detected within a set time period.

4. The method of claim 2, further comprising:
receiving a user selection of two or more spontaneous breath types from a plurality of spontaneous breath types.

5. The method of claim 4, wherein the two or more selected spontaneous breath types comprise: proportional assist (PA), pressure support (PS) and volume support (VS) breath types.

6. The method of claim 4, further comprising:
detecting a patient effort within a set time period;
comparing the one or more patient respiratory parameters to at least one predetermined spontaneous threshold; and
delivering a spontaneous breath of a selected one of the two or more selected spontaneous breath types to the patient based on results of the spontaneous comparing operation.

7. The method of claim 6, wherein the at least one predetermined spontaneous threshold is at least one of work of breathing, patient effort, carbon dioxide, inspiratory pressure, expiratory pressure, respiratory rate, inspiratory volume, expiratory volume, body weight, minute ventilation, lung/chest wall compliance, tidal volume, airway resistance, PaCO₂, SO₂, peak inspiratory pressure, and target pressure threshold.

8. The method of claim 1, wherein the one or more patient respiratory parameters comprise: work of breathing, patient effort, carbon dioxide, inspiratory pressure, expiratory pressure, respiratory rate, inspiratory volume, expiratory volume, body weight, minute ventilation, lung/chest wall compliance, tidal volume, airway resistance, PaCO₂, SpO₂, peak inspiratory pressure, and target pressure.

9. The method of claim 1, wherein the two or more selected mandatory breath types comprise: volume control (VC), pressure control (PC), and volume-targeted-pressure-control (VC+) breath types.

10. The method of claim 1, wherein the at least one predetermined mandatory threshold is at least one of work of breathing, patient effort, carbon dioxide, inspiratory pressure, expiratory pressure, respiratory rate, inspiratory volume, expiratory volume, body weight, minute ventilation, lung/chest wall compliance, tidal volume, airway resistance, PaCO₂, SpO₂, peak inspiratory pressure, and target pressure threshold.

11. The method of claim 1, wherein the one or more patient respiratory parameters are monitored during exhalation.

12. The method of claim 1, wherein the one or more patient respiratory parameters are monitored during inhalation and exhalation.

13. A ventilator system, comprising:
at least one processor; and
at least one memory, communicatively coupled to the at least one processor and containing instructions for a plurality of breath types and instructions for operating a ventilator in at least an adjusting mandatory mode that, when executed by the at least one processor, perform a method comprising:
monitoring one or more patient respiratory parameters during ventilation of a patient;
comparing at least one monitored patient respiratory parameter to at least one predetermined mandatory threshold;
selecting one of a preselected set of mandatory breath types based on results of the mandatory comparing operation; and
delivering a mandatory breath of the selected one of the preselected set of mandatory breath types to the patient.

14. The method of claim 13, wherein the one or more patient respiratory parameters are monitored during exhalation.

15. The method of claim 13, wherein the one or more patient respiratory parameters are monitored during inhalation and exhalation.

16. The method of claim 13, wherein the instructions for operating the ventilator include instructions for operating the ventilator in an Adjusting Hybrid Mode that, when executed by the at least one processor, further performs a method comprising:
detecting a patient effort within a set time period; and
delivering a spontaneous breath of a preselected spontaneous breath type to the patient.

17. The method of claim 13, wherein the instructions for operating the ventilator include instructions for operating the ventilator in an Adjusting Hybrid Mode that when executed by the at least one processor, further performs a method comprising:
detecting a patient effort within a set time period;
comparing the at least one monitored patient respiratory parameter to at least one predetermined spontaneous threshold;
selecting one of a preselected set of spontaneous breath types based on results of the spontaneous comparing operation; and
delivering a spontaneous breath of the selected one of the preselected set of spontaneous breath types to the patient.

18. The method of claim 17, wherein the preselected set of spontaneous breath types comprise: proportional assist (PA) and volume support (VS) breath types.

19. The method of claim 17, wherein the at least one predetermined spontaneous threshold is at least one of work of breathing, patient effort, carbon dioxide, inspiratory pressure, expiratory pressure, respiratory rate, inspiratory volume, expiratory volume, body weight, minute ventilation, lung/chest wall compliance, tidal volume, airway resistance, PaCO₂, SpO₂, peak inspiratory pressure, and target pressure threshold.

20. The method of claim 13, wherein the one or more patient respiratory parameters comprise: work of breathing, patient effort, carbon dioxide, inspiratory pressure, expiratory pressure, respiratory rate, inspiratory volume, expiratory volume, body weight, minute ventilation, lung/chest wall compliance, tidal volume, airway resistance, PaCO₂, SpO₂, peak inspiratory pressure, and target pressure.

21. The method of claim 13, wherein the preselected set of mandatory breath types comprise: pressure control (PC), volume control (VC), and volume-targeted-pressure-control (VC+) breath types.

22. The method of claim 13, wherein the at least one predetermined mandatory threshold is at least one of work of breathing, patient effort, carbon dioxide, inspiratory pressure, expiratory pressure, respiratory rate, inspiratory volume, expiratory volume, body weight, minute ventilation, lung/chest wall compliance, tidal volume, airway resistance, PaCO₂, SpO₂, peak inspiratory pressure, and target pressure threshold.

23. A graphical user interface for a ventilator, the ventilator configured with a computer having a user interface including the graphical user interface for accepting commands, the graphical user interface comprising:
at least one window associated with the graphical user interface;
one or more elements within the at least one window, comprising at least one of:
a mode button allowing selection of one of a plurality of modes; and
a mandatory breath type selection element through which a plurality of mandatory breath types is selected to be delivered to a patient.

24. The graphical user interface of claim 23, wherein the one or more elements within the at least one window, further comprises at least one of:
a spontaneous breath type selection element through which a plurality of spontaneous breath types is selected to be delivered when the ventilator is delivering a breath in response to detection of a patient effort within a set time period.

25. The graphical user interface of claim 23, wherein a mode of the plurality of modes comprises at least one of an Adjusting Mandatory Mode, an Adjusting Spontaneous Mode, and an Adjusting Hybrid Mode.

26. The graphical user interface of claim 25, wherein the one or more elements within the at least one window, further comprises at least one of:
a setting window after the selection of the Adjusting Hybrid mode, the setting window allowing selection of at least one of a mandatory adjust setting, a spontaneous adjust setting, and a dual adjust setting.
